(19) United States
(12) Patent Application Publication
(10) Pub. No.: US 2026/0000796 A1
(43) Pub. Date: Jan. 1, 2026

(54) UVC HAND DISINFECTION DEVICE

(71) Applicant: Holy Spirit University of Kaslik, New York, NY (US)

(72) Inventor: Joseph DGHEIM, Jounieh (LB)

(73) Assignee: Holy Spirit University of Kaslik, New York, NY (US)

(21) Appl. No.: 18/754,910

(22) Filed: Jun. 26, 2024

(51) Int. Cl. A61L 2/00; A61L 2/24; A61L 2/26

(52) U.S. Cl. CPC A61L 2/0047; A61L 2/24; A61L 2/26; A61L 2202/11; A61L 2202/122; A61L 2202/14

(57) ABSTRACT

A hand disinfection device (HDD) that includes a housing having a least one opening that is sized to permit entry of a human hand into the housing; and two oppositely disposed UV lamps that are spaced far enough to permit the human hand to be located therebetween without making direct contact with the UV lamps each emitting UVC in the wavelength range 207-222 nanometers

### FIELD OF THE INVENTION

The present invention relates to an ultra-violet hand disinfection device.

### BACKGROUND OF THE INVENTION

Deadly infectious diseases may be spread through direct contact with contaminated hands. In the healthcare service environments contaminated hands can have severe consequences for the patients and the healthcare providers.

Centers for Disease Control and Prevention (CDC) provides extensive guidelines on infection control, differentiating between sterilization and disinfection. Sterilization is defined as the process of killing or removing all forms of microbial life, including bacterial spores, which is achieved through application of heat, chemicals, or radiation, for example. Disinfection does not necessarily kill all forms of microorganisms, particularly resistant bacterial spores; it is aimed at reducing the number of viable microorganisms to a level that is not harmful to health. World Health Organization (WHO) guidelines also differentiate between the two processes, noting that disinfection refers to processes that eliminate many or all pathogenic microorganisms, except for bacterial spores, on inanimate objects. Rutala W A, Weber D J, Healthcare Infection Control Practices Advisory Committee (HICPAC) guidelines define the levels of disinfection and sterilization required for different medical instruments and surfaces, indicating that the terminology depends on the degree of microbial kill.

Hand disinfection is crucial to preventing the transmission of pathogens.

There are conventional methods of disinfection. However, many microorganisms are becoming resistant to conventional disinfection methods.

It has been recognized that hand disinfection using UVC can be efficient and convenient.

UVC light has germicidal properties, meaning it can inactivate and/or destroy bacteria and viruses present on the skin's surface.

When, for example, bacteria are exposed to UVC light, chemical reactions take place that mutate the genetic material (DNA/RNA) of the bacteria rendering the bacteria harmless bacteria and/or limiting the growth of the bacteria. Specifically, when UVC reaches the nucleic acids after penetrating the cell wall, adjacent thymine undergo dimerization, which causes a kink or a bend in the DNA strand preventing DNA replication.

Light is strongly absorbed by biological materials. Consequently, light does not have the ability to penetrate even the outermost layers of human skin and the outer tear layer of the eye, both of which lack living cells. However, since bacteria and viruses are typically much smaller in size, far-UVC light can still efficiently pass through and deactivate them. In this respect, the 254 nm emission of low-pressure vacuum lamps is known to have antimicrobial effect. However, 254 nm light is also harmful to human cells.

It has been discovered that far UVC has a spectral region of 200-230 nm that can inactivate pathogens without harming human cells if the exposure time and dosage are correctly selected.

It has also become known that 222 nm light is particularly suited for disinfection without harming human cells when the exposure time and dosage are correctly limited.

### SUMMARY OF THE INVENTION

It is an object of the present invention to provide a device for disinfection of human skin using far UVC in the range 200-230 nm, in particular 222 nm.

A device according to the present invention has a stainless steel housing with one or more openings for hand reception.

The housing is occupied with two far UVC lamps that emit 207 to 222 (preferably 222) nm light for disinfection. Far-UVC light having a wavelength in the range 207 nm and 222 nm effectively kills drug-resistant bacteria while showing no apparent harm to exposed mammalian skin because light is strongly absorbed by biological materials, which limits the ability of the light to penetrate even the outermost layers of human skin. However, since bacteria and viruses are typically much smaller in size, far UVC light can still efficiently pass through and deactivate them. Unlike 254 nm light, which is not suitable for use in occupied spaces due potential for harm to human health, far-UV light at 222 nm opens up the possibility of disinfecting hands in areas where people are present, such as hospitals, public transportation, or offices because the far-UV light at 222 nm is still effective at damaging the DNA or the RNA of microorganisms, while minimizing the impact on human cells. This targeted approach enhances the efficiency of the disinfection process.

A hand disinfection device (HDD) according to the present invention includes a housing having a least one opening that is sized to permit entry of a human hand into the housing; and two oppositely disposed UV lamps that are spaced far enough to permit the human hand to be located therebetween without making direct contact with the UV lamps each emitting UVC in the wavelength range 207-222 nanometers, preferably 222 nanometer.

The housing may have a single opening sized to permit entry of the human hand into the housing, or two spaced openings each sized to permit entry of the human hand into the housing.

The UV lamps may be Krypton Chloride excimer lamps.

The HDD may further include a UV transparent guard mounted inside the housing between the UV lamps arranged to prevent direct contact between the human hand and the UV lamps. The UV transparent guard may be a grid panel.

The HDD may further include a first mirror on an interior surface of the housing opposite one UV lamp, and a second mirror on another interior surface of the housing opposite the other UV lamp. The mirrors may be aluminum-covered mirrors.

The HDD may further include UV shields mounted inside the housing and positioned at each opening to reduce or eliminate the possibility of UV light escaping out of the housing.

Each UV shield may include a first portion and a second portion each made of a flexible body, the first and the second portions being positioned adjacent one another along respective edges thereof.

Each shield portion may instead be a brush with flexible bristles that allow the entry and withdrawal of a human hand into and out of the housing.

The HDD may further include a circuit board mounted inside the housing, the circuit board supporting components for controlling the operation of the UV lamps.

The HDD may further include a warning feature controlled by the circuit board that generates sound or light to indicate exposure time. The warning feature may be a buzzer to generate sound, a light source, or a display that displays exposure time.

The circuit board may control the proper dose and proper exposure time to UVC to perform disinfection without causing cellular or tissue damage to the human hand.

The HDD may further include a DC power supply that includes a rechargeable battery.

The HDD may further include at least one proximity sensor mounted inside the housing to sense the entry of the human hand into the space between the two UV lamps and to generate a signal to activate the UV lamps for a predetermined period of time.

Other features and advantages of the present invention will become apparent from the following description of the invention which refers to the accompanying drawings.

### BRIEF DESCRIPTION OF THE DRAWINGS

FIG. 1 shows a bottom view of a device according to the present invention.

FIG. 2 shows a top view of the device of FIG. 1 with the top side of its housing rendered transparent to show the components therein.

FIG. 3 shows a side view of the device of FIG. 1 with a side of its housing rendered transparent to show the components therein.

FIG. 4 schematically shows the components of the electronic circuit components of a device according to the present invention.

### DETAILED DESCRIPTION OF EMBODIMENTS OF THE INVENTION

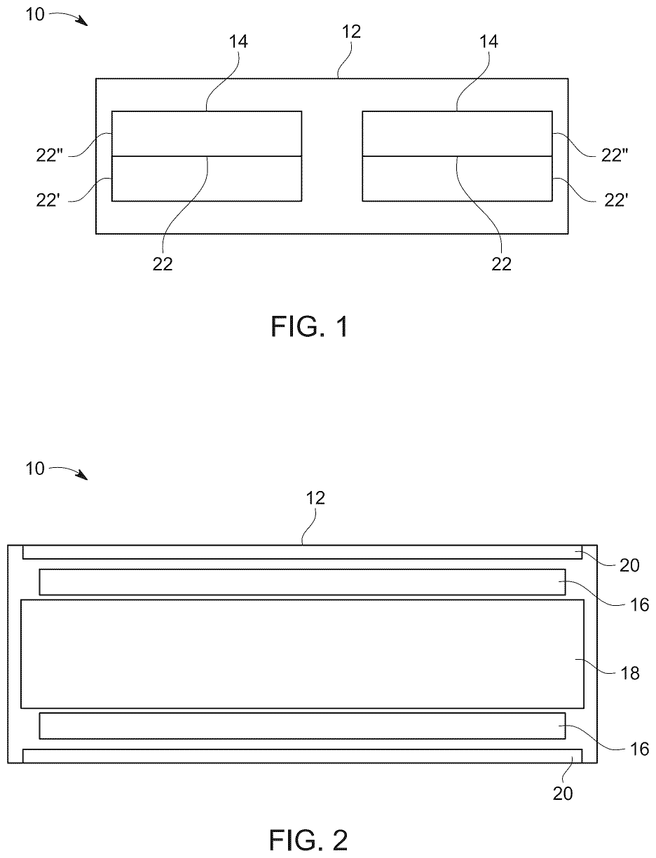

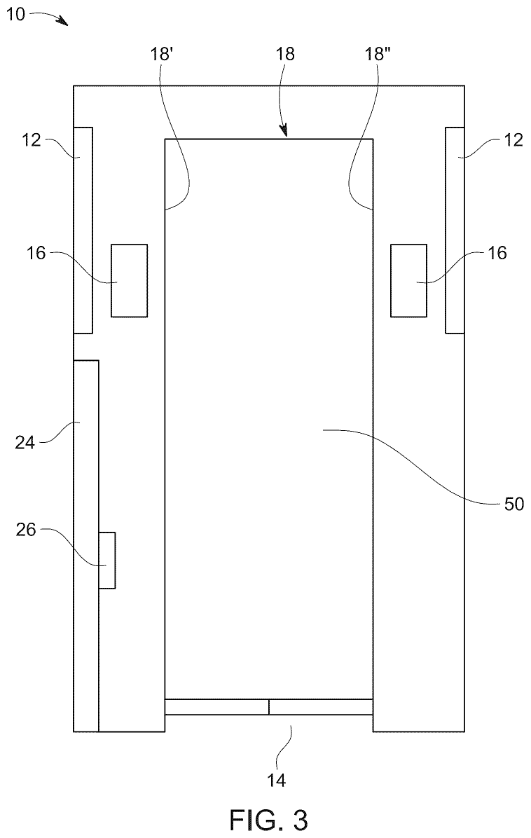

Referring to FIGS. 1, 2 and 3, a Hand Disinfection Device (HDD) 10 according to the present invention includes a housing 12. The housing 12 includes, for example, two spaced openings 14 each sized to permit the entry of a human hand into the housing 12. It should be noted that instead of two openings 14 a single opening may be provided that is sized to permit the simultaneous entry of two human hands into the housing 12.

The HDD 10 further includes two oppositely disposed UV lamps 16 (mounted inside the housing 12), which may be Krypton Chloride (KrCl) excimer lamps. The UV lamps 16 are spaced far enough to permit a human hand to be located therebetween without making contact with the UV lamps 16.

A UV transparent guard 18 (for example, a grid panel with openings that permit the passage of UV light) may be mounted inside the housing 12 between the UV lamps 16 to prevent direct contact between the human hand and the UV lamps 16. While the guard 18 may be a single body that receives the human hand between two opposing portions 18′, 18″ thereof, one could have two guards 18 that are discrete bodies and each located to prevent direct contact between the human hand and a respective UV lamp 16. The guard 18 grid also helps to contain and redirect UVC light towards the disinfection zone 50 between the two UV lamps 16. The housing 12 is made of a material that is opaque to the UV light emitted by UV lamps 16. For example, the housing 12 may be made of stainless steel.

In addition, the HDD 10 according to the present invention may have a first mirror 20 on an interior surface of the housing 12 opposite one UV lamp 16, and a second mirror 20 on another interior surface of the housing 12 opposite the other UV lamp 16. The mirrors 20 are mounted inside the housing 12 to reflect UV light from the UV lamps 16 in a direction away from the interior walls and toward the center of the housing 12 particularly the disinfection zone 50. The mirrors 20 may be aluminum-covered mirrors, and provide additional safety to the user.

The HDD 10 may further include UV shields 22 (mounted inside the housing 12) positioned at each opening 14 to reduce or eliminate the possibility of UV light escaping out of the housing 12 when the device is in use.

Each shield 22 includes a first portion 22′ and a second portion 22″, preferably mounted inside the housing 12. Portions 22′, 22″ are made of a flexible, for example, flat body. Portions 22′,22″ are positioned adjacent and preferably in contact with one another along respective edges thereof. A user's hand enters the housing 12 by passing between the contacting edges of the first portion 22′ and the second portion 22″ and deforming the two portions 22′,22″ to open just enough space to allow the passage of the entering hand while minimizing the escape of UV light. Each shield portion 22′, 22″ may be in the form of a brush with flexible bristles that allow the entry and withdrawal of a human hand into and out of the housing 12. The brushes have enough bristles to enable their function as a protective barrier preventing unintended leakage of UV light that may expose nearby persons. Also, the brushes contribute to maintaining a clean environment within the housing 12 by reducing/preventing the entry of dust, debris, or other contaminants into the housing 12. The brushes are also disinfected by UVC light.

The HDD 10 further includes a circuit board 24 (mounted inside the housing 12) that supports components for controlling the operation of the UV lamps 16. The circuit board 24 may be positioned on one of the interior surfaces of the housing 12.

A proximity sensor 26 may be provided (mounted inside the housing 12) to sense the entry of a human hand into the space between the two lamps and to generate a signal to activate the UV lamps 16 for a predetermined period of time. For example, the proximity sensor 26 may be located centrally so that it may sense the entry of either the left hand or the right hand into the housing and between the UV lamps 16. Instead of one proximity sensor 26, two proximity sensors 26 may be provided, one for sensing the entry of the left hand, and the other for sensing the entry of the right hand into the housing 12 so that the entry of either hand may lead to the activation of the UV lamps 16. For additional safety, the UV lamps 16 will remain ON only if the presence of at least one human hand inside the housing 12 is detected by the proximity sensor(s) 26. That is, when the presence of a human hand is not detected, the UV lamps 16 are shut off. Alternatively, the UV lamps 16 may be shut off under the control of the circuit board 24 after a predetermined amount of time has passed after the detection of the entry of a human hand into the housing 12.

The HDD 10 is devised to address the technical problem of repeat exposures to UVC, which affects the superficial layers of skin, mucous membranes and eye tissues. That is, as controlled by the circuit board 24, the HDD 10 provides the proper dose and proper exposure time to UVC to perform disinfection without causing cellular or tissue damage to the human hand with a far UV emission in the wavelength range 207-222 nanometers, preferably 222 nm.

Optionally, the HDD 10 may be further equipped with a warning feature that generates sound (e.g. a buzzer), and/or emits light (e.g. green light changing to red light to instruct the user to withdraw his/her hands), and/or displays effective exposure time (in seconds) to the user. The warning feature may be controlled by the circuit board 24 and placed anywhere that is most convenient. For example, in case of light or exposure time, the warning feature may be placed somewhere on the front side of the exterior of the housing 12 facing the user. In the event the warning feature is a sound generator such as a speaker, it could be placed anywhere except the side of the housing 12 used for mounting the housing 12 to a wall.

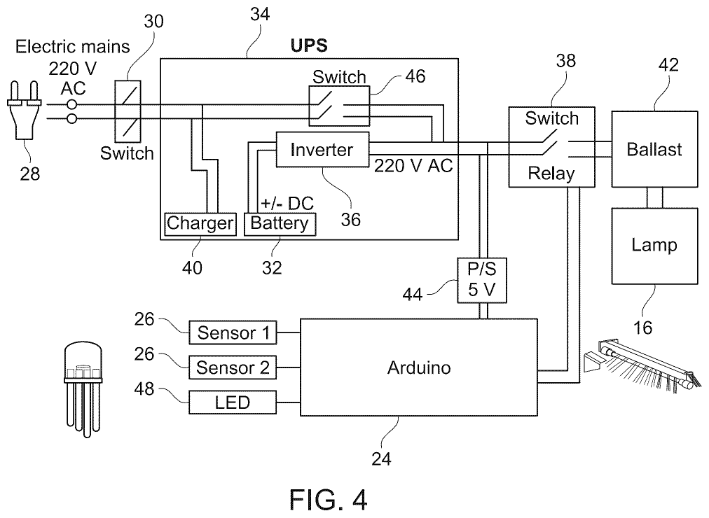

Referring to FIG. 4, the HDD 10 may receive line power via a power plug 28 inserted in a power supply socket that supplies electric power (e.g. 110 V or 220 V). The line power may be switched ON/OFF by a switch 30. The power so supplied may charge a battery 32 in an uninterrupted power supply (UPS) 34. The line power, while being supplied, will bypass an inverter 36 and continue normally to a relay 38, and a charging circuit 40 will charge the battery 32 simultaneously. When power from the line is interrupted, the battery 32 will provide DC power which is inverted by the inverter 36 to provide the appropriate AC voltage (e.g. 110 V or 220V) output. The AC output will be delivered to the relay 38 which will distribute electricity to a ballast 42 that operates the UV lamps 16. The relay 38 may be controlled by the circuit board 24 (which may be an Arduino board) functioning as the brain of the system. Since the circuit (Arduino) board 24 needs 5V to function a 5V power supply 44 is provided and connected to it. A switch 46 may be provided inside the UPS 34 to disconnect direct line power connection. An LED 48 that changes color (from green to red and red to green for example) may be provided as a warning feature.

In summary, the HDD 10 may have the following features:
two 222 nm UVC lamps;
a buzzer for sound indication during disinfection;
RGB LED to signal the start and end of hand disinfection;
mirrors to reflect light onto the sides of the hands;
two motion sensors to detect hand placement;
upside brushes and/or a novel bouncing design on the openings to limit external UVC radiation and to ensure the user's hands are positioned equidistantly between both lamps;
adjustment of time/distance settings using real experimental data;
compact, portable, and/or fixed design;
integratable into hot air dryers; and
a DC power supply in case of electrical current cutoff.

The UVC hands disinfection machine is highly effective in eliminating a wide range of microorganisms, including bacteria and viruses, from the surfaces of hands. However, it is essential to distinguish between disinfection and sterilization. Sterilization typically involves the complete destruction or elimination of all forms of microbial life, including spores, and viruses; whereas, disinfection reduces the microbial load to a level considered safe for public health. While the HDD 10 achieves a high level of disinfection, it should not be understood that the HDD 10 must meet the stringent criteria required for sterilization because sterilization standards typically demand a higher level of microbial reduction, including the elimination of spores and viruses which may not be achievable solely through UVC irradiation on hands.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

What is claimed is:

1. A hand disinfection device (HDD), comprising:
a housing having a least one opening that is sized to permit entry of a human hand into the housing; and
two oppositely disposed UV lamps that are spaced far enough to permit the human hand to be located therebetween without making direct contact with the UV lamps each emitting UVC in the wavelength range 207-222 nanometers.

2. The HDD of claim 1, wherein the housing has a single opening sized to permit entry of the human hand into the housing, or two spaced openings each sized to permit entry of the human hand into the housing.

3. The HOOD of claim 1, wherein the UV lamps are Krypton Chloride excimer lamps.

4. The HDD of claim 1, further comprising a UV transparent guard mounted inside the housing between the UV lamps arranged to prevent direct contact between the human hand and the UV lamps.

5. The HDD of claim 4, wherein the UV transparent guard comprises a grid panel.

6. The HDD of claim 1, further comprising a first mirror on an interior surface of the housing opposite one UV lamp, and a second mirror on another interior surface of the housing opposite the other UV lamp.

7. The HDD of claim 1, wherein the mirrors are aluminum-covered mirrors.

8. The HDD of claim 1, further comprising UV shields mounted inside the housing and positioned at each opening to reduce or eliminate the possibility of UV light escaping out of the housing.

9. The HDD of claim 8, wherein each UV shield includes a first portion and a second portion each made of a flexible body, the first and the second portions being positioned adjacent one another along respective edges thereof.

10. The HDD of claim 8, wherein each shield portion is a brush with flexible bristles that allow the entry and withdrawal of a human hand into and out of the housing.

11. The HDD of claim 1, further comprising a circuit board mounted inside the housing, the circuit board supporting components for controlling the operation of the UV lamps.

12. The HDD of claim 11, a warning feature controlled by the circuit board that generates sound or light to indicate exposure time.

13. The HDD of claim 12, wherein the warning feature is a buzzer to generate sound.

14. The HDD of claim 12, wherein the warning feature is a light source.

15. The HDD of claim 12, wherein the warning feature is a display that displays exposure time.

16. The HDD of claim 11, wherein the circuit board controls proper dose and proper exposure time to UVC to perform disinfection without causing cellular or tissue damage to the human hand.

17. The HDD of claim 1, wherein the UV lamps emit UVC with a wavelength of 222 nm.

18. The HDD of claim 1, further comprising a DC power supply that includes a rechargeable battery.

19. The HDD of claim 1, further comprising at least one proximity sensor mounted inside the housing to sense the entry of the human hand into the space between the two UV lamps and to generate a signal to activate the UV lamps for a predetermined period of time.
